(19) United States
(12) Patent Application Publication
(10) Pub. No.: US 2025/0319321 A1
(43) Pub. Date: Oct. 16, 2025

(54) THERAPEUTIC SYSTEM PROVIDING BALANCED ENERGY TO A USER

(71) Applicant: Jason Kruk, Stamford, CT (US)

(72) Inventor: Jason Kruk, Stamford, CT (US)

(21) Appl. No.: 19/097,930

(22) Filed: Apr. 2, 2025

Related U.S. Application Data
(60) Provisional application No. 63/633,089, filed on Apr. 12, 2024.

(51) Int. Cl. A61N 5/00; A61N 5/06

(52) U.S. Cl. CPC A61N 5/00; A61N 2005/0626; A61N 2005/0632; A61N 2005/0644; A61N 2005/0667

(57) ABSTRACT

A therapeutic system including a generator for outputting energy at a first frequency; and at least one probe having a skin contacting surface the probe electrically connected to the generator. The generator transmits energy to the user at the first frequency via the at least one probe and energy from the user is returned to the generator via the at least one probe, and subsequently energy is transmitted back from the generator to the user via the at least one probe.

### BACKGROUND OF THE INVENTION

This application claims priority to provisional application Ser. No. 63/633,089, filed Apr. 12, 2024, the entire contents of which are incorporated herein by reference.

### FIELD OF THE INVENTION

This application is directed to a system for transmitting pathological energies into the body of the user as a balanced state of physiological energy.

### BACKGROUND

Scalar wave technology is used by certain health care practitioners to provide penetration of scalar waves into the cells of the body with the intent to restore cellular voltage and enhance mitochondrial function, leading to increased energy levels. Such scalar energy is used for reducing pain and inflammation.

It would be beneficial to enhance the efficacy of scalar wave technology to improve reduction of pain and inflammation and improve adverse health conditions of individuals. It would also be beneficial to widen the use and availability of scalar wave technology.

### SUMMARY OF THE INVENTION

The present invention overcomes the problems and deficiencies of the prior art. The present invention utilizes scalar wave technology and frequency to administer therapeutic solutions to treat psychological and physiological health conditions. Various clinical applications are contemplated as described in detail below.

The present invention, more specifically, provides a system that detects disturbed patterns in the user, which are transmitted to a generator which subsequently outputs rebalanced frequency to the user's body. In some embodiments, balanced energy is also transmitted to a liquid such as purified water via the generator for user consumption. In some embodiments, energy from one or more select remedies are transmitted from the generator to the user's body and/or to a liquid for user consumption. Each of these embodiments/versions are discussed in detail below.

The system, in brief, includes a generator, one or more probes connected to the generator and in contact with the user, and input and output plates connected to, or positioned on, the generator. Each of these features are discussed in detail below.

The system can be used to transmit energy continuously in a continuous therapeutic mode or to transmit pulsed energy in a pulsed therapeutic mode.

Data storage capabilities in the system and/or in a remote device are also provided in some embodiments. Data can in some embodiments be provided to a clinician, technician, health care professional, etc. monitoring usage of the system and user reactions. In some embodiments, the data stored can include initial and subsequent energy levels, e.g., frequency, vibration, time periods of energy application, etc. User reaction can also be quantified for evaluation and feedback to the user and/or third party, such as a health care provider.

The generator in some embodiments is configured for activation by a start button on the generator. In alternate embodiments, the generator can be activated by a remote device in cable/wired connection or in wireless communication for remote activation. Voice activation of the generator is also contemplated.

In some embodiments, the generator can include a graphic display or be in wireless communication with a graphic display to display various aspects/features of the system as described below.

In some embodiments, data is collected and stored of parameters of use of the device. The data can be stored on one or both of the system itself and/or an external device. The external storage device could also be the device for controlling energy applications.

In some embodiments, the device is configured to be in communication with a Mobile App running on an external smartphone, or software running on a personal computer, which captures various features.

In accordance with one aspect of the present invention, a therapeutic system is provided comprising a generator for outputting energy at a first frequency and at least one probe electrically connected to the generator, the at least one probe having a skin contacting surface. The generator transmits energy to the user at the first frequency via the at least one probe and energy from the user is returned to the generator via the at least one probe, and subsequently energy is transmitted back from the generator to the user via the at least one probe.

In some embodiments, the at least one probe comprises first and second hand probes engageable by respective hands of the user and/or first and second foot probes engageable by a respective foot of the user.

In some embodiments, energy from the user sensed as unbalanced or disturbed is changed into a balanced state and balanced energy is sent back to the user via the at least one probe. In some embodiments, the generator includes one or more filters to change energy into the balanced state. The energy can be pulsed or transmitted continuously.

In some embodiments, the system further includes a data storage device for storing data relating to one or more of a) usage of the system; b) parameters during use of the system; and/or c) reactions by the user of the system. The data storage device can be in the generator or a separate component in wired or wireless communication with the generator.

In some embodiments, the generator is activated by a remote device.

In some embodiments, the therapeutic system further includes a graphic display displaying parameters during use of the system.

In some embodiments, the system includes an outplate (output plate) in communication with the generator. The outplate configured to communicate with a drinkable liquid in a container (holder) supported on the outplate, wherein the balanced energy is transmitted to the liquid in the container, the liquid providing a therapeutic liquid drinkable by the user. The system can further include in some embodiments an inplate (input plate) configured to support/receive a substance or remedy, and the system detects a vibrational frequency of the substance placed thereon, wherein energy from the substance or remedy on the inplate is combined with the balanced energy and transmitted via the generator to the liquid in the container. The combined energy can be transferred to the user as the user engages the at least one probe, thereby providing therapy to the user via the at least one probe and through a drinkable therapeutic solution. That is, balanced energy is transmitted to the drinkable liquid in communication with the generator to create a therapeutic drinkable liquid.

The system preferably applies scalar energy transmitting scalar waves which characteristics provide therapeutic benefits.

In accordance with another aspect of the present invention. a therapeutic method is provided for transmitting energy to a body of a user to improve health conditions. The method includes a) outputting energy from a generator at a first frequency; b) transmitting the energy at the first frequency to the user via at least one probe electrically connected to the generator, the at least one probe having a skin contacting surface; c) detecting via the at least one probe disturbed energy patterns in the user; d) transmitting the disturbed energy to the generator; e) filtering the disturbed energy in the generator to balance the energy; f) transmitting the balanced energy from the generator to the user via the at least one probe. In some methods, steps e-f are repeated.

In some embodiments, the method further comprises transmitting the balanced energy to a drinkable liquid to provide a therapeutic solution drinkable by the user.

In some embodiments, the method further comprises detecting via a sensor a frequency of the substance or remedy and communicating the frequency of the substance or remedy to the generator communicating with a sensing device.

In some embodiments, the method further comprises combining the energy from the substance or remedy with the balanced energy and transmitting the resulting energy to the user.

In some embodiments, the method further comprises combining the energy from the substance or remedy with the balanced energy and transmitting it to the drinkable liquid.

### BRIEF DESCRIPTION OF THE DRAWINGS

So that those having ordinary skill in the art to which the subject invention appertains will more readily understand how to make and use the system and apparatus (device) disclosed herein, preferred embodiments thereof will be described in detail hereinbelow with reference to the drawings, wherein:

FIG. 1 is a top perspective view of the generator and hand and foot probes of the system of an embodiment of the present invention;

FIG. 2 is a top perspective view of the generator of FIG. 1 showing the input and output plates in accordance with embodiments of the present invention;

FIG. 3 is a flow diagram illustrating the overall system of the present invention wherein the generator transmits energy to the user via hand and/or foot probes, and energy is transmitted back to the generator, and then energy is returned to the individual in a circular loop;

FIG. 4 is a flow diagram of an alternate embodiment of the system of the present invention showing the initial energy output from the generator followed by modified output from the generator in response to vibrational patterns of the user for therapeutic treatment, and further showing a liquid container on the output plate;

FIG. 5 is a flow diagram of the embodiment of the system wherein energy is additionally outputted to a liquid, e.g., water, on the outplate for consumption by the user; and

FIG. 6 is a flow diagram of an alternate embodiment of the system of the present invention wherein energy is additionally outputted to a liquid, e.g., water, for consumption by the user, and energy from a substance or remedy on the input is further transmitted to the liquid on the outplate and to the user.

### DETAILED DESCRIPTION OF PREFERRED EMBODIMENTS

The present invention is directed to a therapeutic system utilizing vibrational energy to achieve a more balanced state of the user. In general, the system includes a generator, probe(s) to transmit energy from the generator to the user and from the user back to the generator and then back to the user in a circular loop to optimize energy balance of the user.

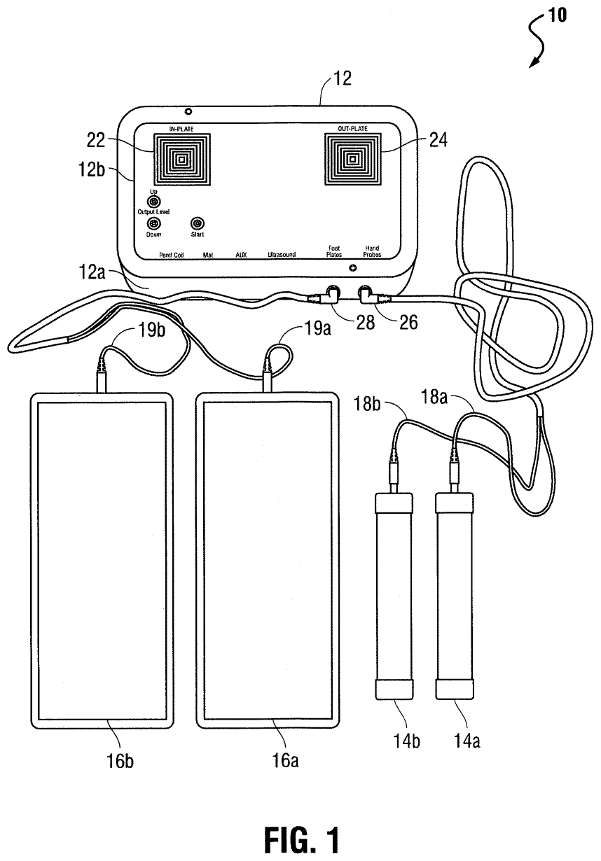

Referring now to the drawings and particular embodiments of the present disclosure, wherein like reference numerals identify similar structural features or methods of the systems throughout the several views, FIG. 1 illustrates the components of an embodiment of the system. The system, designated generally by reference numeral 10, includes a generator 12, a pair of hand probes 14a, 14b (collectively hand probes 14) to be held in the user's right and left hands, respectively, and a pair of foot probes 16a, 16b (collectively foot probes 16) for the user's right and left foot, respectively. The hand probes 14a, 14b are connected to the generator 12 by cables or wires 18a, 18b, respectively, although other forms of connectors as well as wireless connections, e.g., Bluetooth, NFC, etc. are also contemplated. The foot probes 16a, 16b are connected to the generator 12 by cables or wires 19a, 19b, respectively, although other forms of connectors as well as wireless connections, e.g., Bluetooth, NFC, etc. are also contemplated.

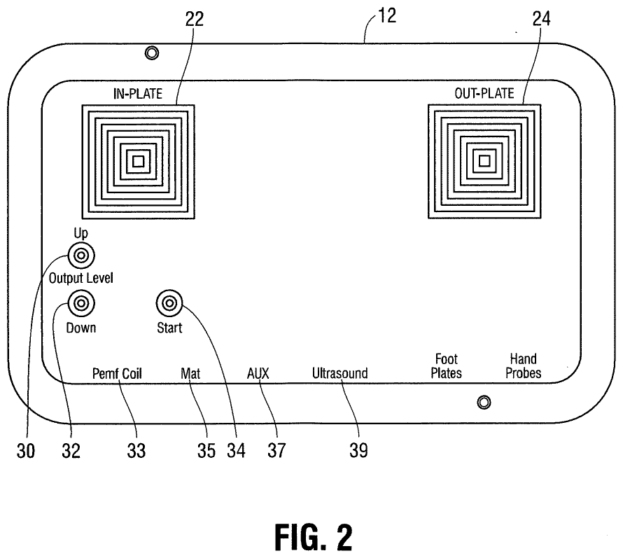

With reference to FIGS. 1 and 2, generator 12 includes an inplate 22 (also referred to herein as an input plate) and an outplate 24 (also referred to herein as an output plate). The inplate 22 and outplate 24 are shown square shaped, but other sizes as well as other shapes/configurations are also contemplated, and the configurations/shapes and sizes of the input plate and output plates can be the same or different. Also, although the inplate plate 22 is shown on the left side and the output plate 24 is shown on the right side, the positioning could be reversed so that inplate plate 22 is on the right side and the output plate 24 is on the left side. Additionally, the plates 22, 24 can be positioned on other locations of the generator. It is also envisioned that the output plate and/or input plate can be separate components attached to the generator, either via wired or wireless communication. More than one output plate and/or more than one input plate is also contemplated.

Generator 12 also includes a connection port 26 for the hand probes 14 and a connection port 28 for the foot probes 16, shown in a side wall 12a of the generator 12. Further generator controls include an output level which can be adjusted up (reference numeral 30) or down (reference numeral 32) and a start button 34 on a top wall 12b of generator 12. The generator 12 can also have outputs 33 and 35 for Pulsed Electro-Magnetic Field (PEMF) coils and mats. The generator can also have an output 37 for light therapy and an output 39 for ultrasound/sound frequency therapy. Note the locations of the connection ports, controls, start button, outputs, etc. in FIG. 2 are shown by way of example, it being understood that the locations can be different than that shown in FIG. 2 and fewer or more controls can be provided dependent on use. Also, instead of buttons, other mechanisms, e.g., a lever, slidable switch, rocker switch, etc., can be used to activate the system. The various features of the generator 12 can include LEDs or other light indicators to indicate operation, i.e., turning on, of the generator. The generator 12 can also include a display, e.g. a display screen to show the energy level, or frequency input and/or output to and from the user, or other features/parameters. It should be appreciated that in some embodiments, the generator does not need to have all these inputs and the present invention is not limited to the inplate generator shown in the drawings.

It is also contemplated that the generator can be turned on and/or controlled by a remote computing device such as a smartphone, laptop, etc.

It is also contemplated that data regarding the frequency, period of use, adjustments, etc. can be stored in the generator or a computing device connected either via wires/cable or wirelessly to the generator. Such data can be used to inform the user for future use. Such data can also be used by a health care practitioner to assess treatment efficacy. Machine learning is also contemplated to process the data and provide improved parameters and inputs for the particular user or overall for users of the technology.

The generator can in some embodiments include removable batteries in a compartment in the generator housing which can be charged by a wall adapter, e.g., a smart charger, plugged into the unit at one end and the wall outlet at the other end.

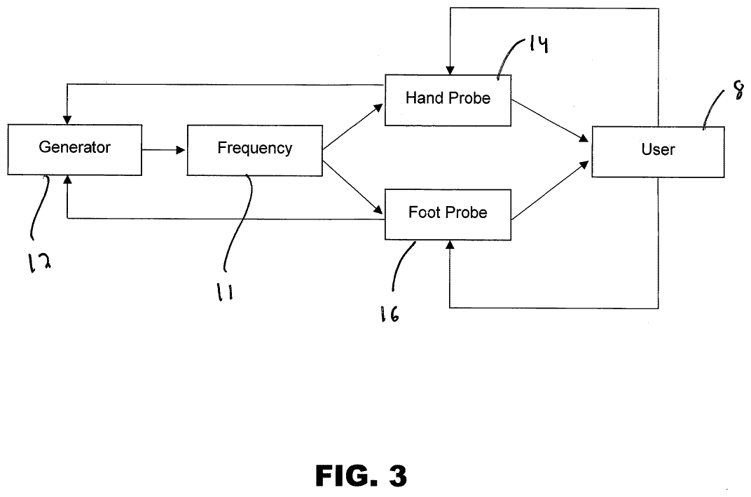

The flow diagram of FIG. 3 illustrates the circular loop of the system. In general terms, the generator 12 initially sends energy to user 8 at a predetermined frequency 11 via the hand probe(s) 14 and foot probe(s) 16. Energy from the user 8 is then returned to the generator 12via the hand probe(s) 14 and foot probe(s) 16 and then back to the user via the probes 14, 16. This can be summarized as follows:

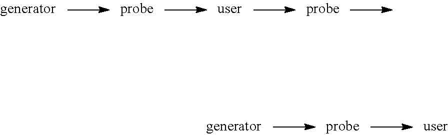

The process is circular, where a user holds with the hands the probes and/or places the feet on the foot pads and the 777 Hz frequency moves through the body and then back into the generator, in a circular field. As the user is feeling vibrational frequency, the modules and filters in the generator sense any unbalanced or disturbed energy or irregular molecular activity in the body, send it back through the generator, filter it into a balanced state, and send the cleaned up vibrational pattern of balance through the body. Without a user, there is a state of balanced 777 Hz frequency. When a user is holding the hand probes or in contact with the foot probes, it essentially aligns any unbalanced or disturbed patterns to a balanced field, resulting in therapeutic applications at the molecular level.

This process is called Physiological Oscillation. Simply put, information (777 Hz) is sent to the body, and information (disturbed patterns, imbalance, illness) is received from the body. This information is then filtered and sent back to the body as a balanced state of physiological energy.

Although both hand probes and foot probes are shown used, in some embodiments only the hand probes or only the foot probes are utilized.

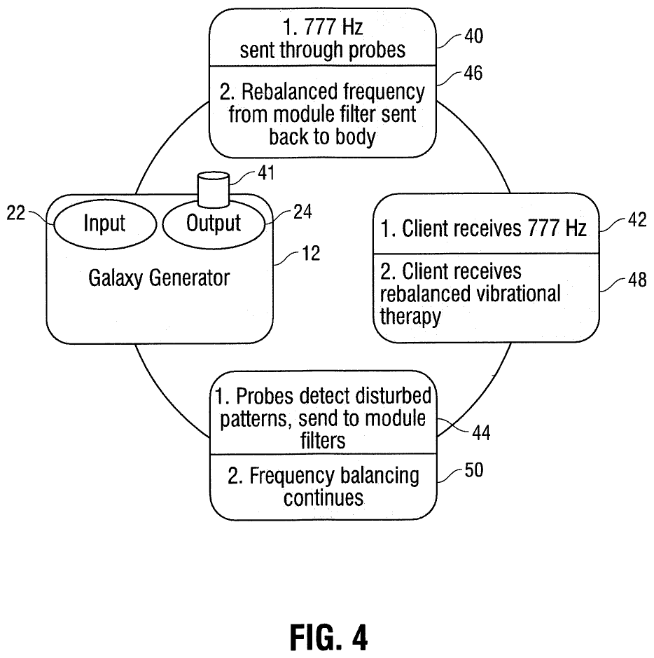

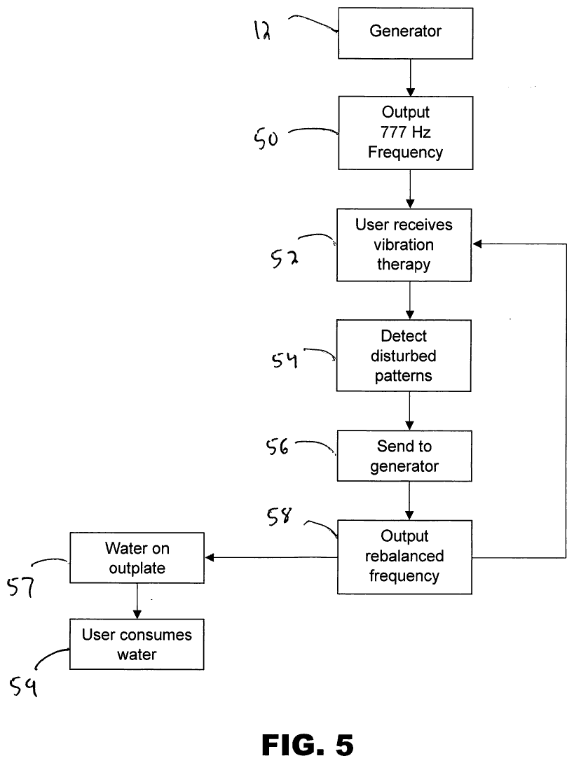

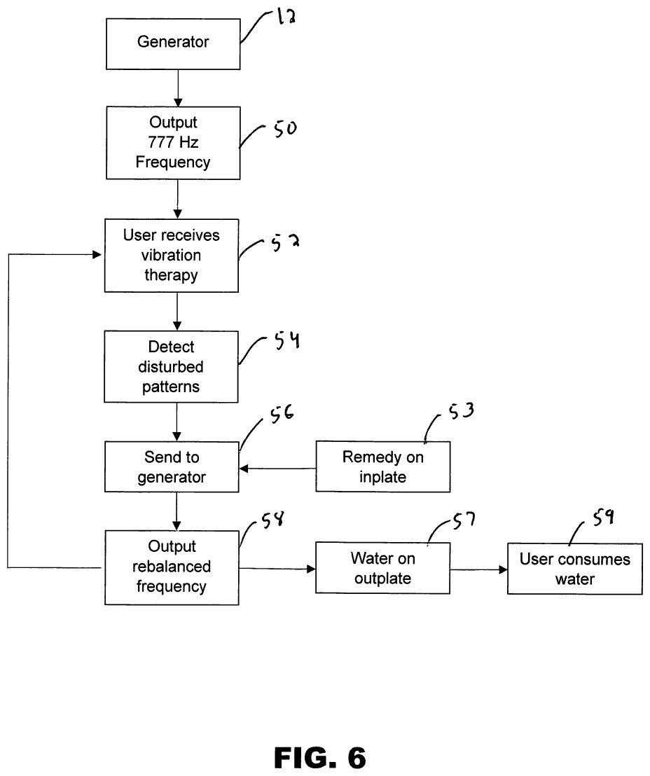

There are three uses of the system of the present invention:
1) System 1: the system of FIG. 3 wherein the balanced energy is inputted from the user into the generator and back to the user;
2) System 2: the system of FIG. 4 (and FIG. 5) which adds to the system of FIG. 3 imbuing a liquid on the outplate with the balanced energy; and
3) System 3: the system of FIG. 6 which adds to the system of FIGS. 4 and 5 imbuing the liquid on the outplate with the energy from a substance/remedy on the inplate.

For convenience, the second system will be referred to herein as the “outplate system” and the third system will be referred to herein as the “inplate/outplate system.” It should be appreciated that these labels are used solely for convenience and should not be interpreted as limiting the scope of the invention.

In each of these systems, as long as the user is holding the hand probes or on the mat (foot probes), magnetic coils, laser light or LED cluster lights are on the body, pathological energies are filtered and sent back into the body as a balanced state of physiological energy.

The outplate system (System 2) can be appreciated by the flow diagram of FIG. 4. Note System 1 can also be considered depicted in FIG. 4 except the outplate would be vacant or not provided. That is, the circular flow of System 1 would be the same as FIG. 4 except an output to a consumable liquid is not included nor is the inplate utilized or provided.

In the outplate system, the user places purified water in a container/bottle on the outplate and holds/engages the hand probes and foot probes/pads. As the user is enjoying the vibrational frequency, the probes sense unbalanced or disturbed energy patterns in the body, and send that information back through the module filters in the generator, clean up those disturbed patterns, purifying them, and send them back into the user's body as a balanced state of energy. At the same time, it is impregnating or imbuing the water molecules in the bottle of purified water on the output plate with the balanced energy. Therefore, the user, while already having received vibrational therapy, can also take the water as an additional therapeutic solution.

More specifically, with reference to FIG. 4, a container 41, such as a bottle, for holding liquid, such as water, is placed on the outplate (output plate) 24 of the generator 12. As shown, the inplate (input plate) 22 is left vacant (or need not be provided) in this embodiment. In the first step 40, 777 HZ frequency is sent by the generator through the probes into the user's body 42. The probes detect disturbed patterns 44 which are sent to the module filters in the generator 12. Rebalanced energy 46 is sent from the module filters back to the user 42 so the user receives rebalanced vibrational therapy 48. The frequency balancing continues 50 as patterns are sent back to the module filters in the generator 12. This circular/continuous loop continues for a designated period of time for achieving the desired therapeutic effects. The container 41 on the outplate 24 contains purified water (or other consumable liquid) which receives electromagnetically charged rebalanced frequency to act as a therapeutic solution which the user can drink.

This system of FIG. 4 can also be appreciated with reference to the flow diagram of FIG. 5. As shown, the generator 12 transmits/outputs 777 HZ frequency 50 to the user 52. Disturbed patterns are detected 54 and sent 56 to the generator 12 which then outputs rebalanced frequency 58 back to the user 52 and at the same time the rebalanced frequency 58 is outputted to the container on the outplate 57 so the therapeutic solution can be consumed by the user 59 after the session.

Time periods of energy application can vary. By way of example, if the user has an inflamed kidney, after holding the hand-held probes for 5-10 minutes, the water becomes a homeopathic restorative substance (made out of the user's own body energy). Sensors within the unit sense disturbed energy patterns in the body which can be remedied of advanced homeopathic preparations.

Various volumes of liquid can be utilized. In one example, a 2 ounce dropper bottle filled with purified water or spring water is used. Drops, e.g., 10 drops by way of example, of peach brandy can be added to the water as a preservative.

Turning now to the inplate/outplate system (System 3), as shown in the flow diagram of FIG. 6, the process is a circular loop as well, with the addition of the inplate remedy's vibrational frequency aligning to the balanced, filtered frequency of the body. That is, a remedy is placed on the input plate which is a sensor plate. The sensor detects the vibrational “essence” or frequency or wave pattern of anything placed on it. When the unit is turned ON, the detected information is run through amplifiers and coils in the generator and transferred to the outplate. This information is transmitted electromagnetically and imprinted into a neutral or blank substance, such as purified water or water and brandy, (e.g. 2 ounces of water), that is placed in a container on the outplate. That is, any substance that is placed on the inplate will oscillate that remedy's vibrational energy through the body as the user holds the probes. This added information combines with the body's new filtered, balanced state and sends that information both through the body and also electromagnetically into the water on the outplate. In this manner, the user (also referred to herein as client) has received two therapeutic modalities: 1) therapy by holding the probes and receiving balanced frequency with the added remedy of whatever is placed on the inplate, and 2) a therapeutic consumable solution imbued on the outplate.

With reference to the diagram of FIG. 6, similar to FIG. 5, generator 60 transmits/outputs 777 HZ frequency 50 to the user 52. Disturbed patterns are detected 54 and sent 56 to the generator 12 which then outputs rebalanced frequency 58 back to the user 52 and at the same time the rebalanced frequency 58 is outputted to the container on the outplate 57 and is consumed by the user 59 after the session. Vibrational energy from the remedy 53 is sent through the generator to the user 52 as well as to the container 57 for consumption.

In this outplate/inplate system, various remedies can be placed on the input plate which will penetrate through the hand/foot probes/electrodes into the body. For example, if the client is needing Vitamin C, calcium or a Bach flower remedy for emotional balancing, a sample of the desired remedy or an appropriate combination of the remedies are placed on the top of the inplate. Other remedies include other vitamins, minerals, herbs, homeopathic remedies, etc. The substance placed on the inplate will oscillate that remedy's energy through the body as the user is holding the probes, plus at the same time the remedy is being charged energetically into the bottle of water. Once the client has received the energy for a predetermined time period, e.g., 5 minutes or more, they can consume the water with the energy from the remedy. Thus, the remedies are made from the body as the water becomes an antidote such as for malfunctions in the brain, for clearing allergies, etc. That is, the therapy becomes impregnated in the water.

Time periods for energy application can vary. By way of example, for certain allergies, 7 drops every 40 minutes has been found to clear up the allergy in four hours (rather than the normal dosage of 7 drops 2-3 times a day).

In summary, the system can be used in three ways: 1. Receive 777 Hz frequency via probes. 2. Receive frequency and put purified water on outplate to receive therapeutic solution. 3. Place remedy on inplate, receive frequency, and output therapeutic solution.

The systems of the present invention use scalar energy, energy formed when two identical frequencies from opposite directions come together and collide, resulting in canceling out of any free movements and the creation of a stationary and static energy field. The scalar waves are two energy fields that are 180 degrees out of phase with each other, when pulsed. In the field of medicine, scalar waves for healing are ideal because medically scalar energy can break down any molecular bonds of fungi and bacteria. Thus, scalar energy can carry information that can be used for healing purposes.

Scalar waves are three-dimensional self-contained waves that spin on one fixed axis. These non-linear waves disseminate throughout the bodies through crystalline lattices of elaborate collagen networks. Scalar waves help in increasing every hydrogen atom's energy covalent level in the body. This is important because hydrogen bonds are crucial to our body's ecosystem as they hold our DNA together. Therefore, scalar waves may be productive in healing at the level of our DNA. It is easier for our bodies to transfer the benefits of scalar energy to our cells, which means it is also easier for scalar energy to transfer scalar waves into the cells, allowing healing at the cellular level.

Scalar waves identify abnormalities in the body and realign them into waves of balance, free from disharmonic waves, illness or decay, transferring that balanced information back into the body. This is accomplished in therapeutic modalities that as explained above include: using hand and/or foot probes to receive scalar wave frequency and/or drinking a therapeutic solution derived from the frequency therapy session.

The positive frequency from items placed on the input plate transmit waves into the body and into the re-balanced frequency created by the body itself and the interaction of the scalar waves. This frequency is in turn, transmitted into a purified water on the output plate, creating a uniquely balanced therapeutic solution. This solution can then be ingested as an additional treatment as it embodies the therapeutic alignment of the scalar wave frequency and re-balanced body alignment.

The scalar wave technology opens up the meridians, acupuncture point, and centers, sometimes referred to as chakras of the body which will allow higher energies to flood the body, which brings healing. It causes the physical body to resonate and realign with the light and etheric body, which is the fullness of divine health. That is, the combination of light and sound, using scalar wave technology opens up portals for higher dimensional energies to flow into the physical dimensions.

The device can be utilized to treat various health conditions. Such conditions can include by way of example extreme nerve pain, hernia, gut pain, diabetes, nearsightedness, cancer, muscle pain and soreness, kidney stones, plantar fasciitis, Parkinson's disease, stroke, optic nerve damage, etc. It can also be used for body relaxation and balance and to treat mental health conditions.

A technician-client scenario will now be explained by way of example:

### EXAMPLE

With the client seated comfortably in front of the generator, place purified drinking water that the client normally drinks throughout the day on top of the right output plate or use a 2-4 ounce dropper bottle of purified water.
Step 1: Before inserting Foot or Hand Probe, press START button on the generator with a quick release. Next, press and hold the DOWN button located below the “Output Level” until the blue light between the Foot and Hand probe jacks is dim or off. Repeat Step 1 each time the unit is turned ON to avoid high output shock.
Step 2: Insert Hand and Foot probes. With the black side up of the foot plates, place bare feet on the black plates and hold the handheld electrodes. Alternatively, a wet paper towel can be placed between the feet and black plates.
Step 3: Press and hold the UP button until desired output level is achieved. To turn unit OFF, press and hold the START button for two seconds. If the client wishes to feel more current, press and hold UP.
Note: At 777 HZ, there is not much current feeling on the foot plates even at 70 Volts. But if the client wishes to pump pulse the 777 HZ into the body, then follow these steps:
Step 4: Disconnect hand and foot probes by pulling the white cable from the jacks.
Step 5: Behind the unit flip up the rocker switch. If the unit is ON and the rocker switch is flipped up, it will automatically turn the unit off to remind you to disconnect the hand and foot probes to void high output shock.
Step 6: Repeat Steps 1, 2 and 3.

Note, in some embodiments, the wall adapter is a smart charger to charge the internal batteries. Insert the wall adapter in the back of the unit and into the wall outlet. When red light on the wall adapter turns green, this indicates the batteries are fully charged. The charger can be plugged in during use or when not in use—the smart charger will not overcharge the batteries.

As noted above, in some embodiments, the system is used continuously (either continuous or pulsed waves) for a predetermined amount of time and the frequencies adjusted as a therapeutic device in a continuous mode. In other embodiments, the system is used intermittently in response to certain input.

In some embodiments, data can be stored/recorded for analysis by the user and/or health care practitioner and adjusted for the particular user for subsequent use. Such data can also be collected from devices used by different users, and the data used and shared among health care professionals or user groups for overall treatment evaluation and advancements. Thus, the data can be stored for data acquisition to provide post-use evaluation. In some embodiments, that data can be evaluated by the user to inform future use. In some embodiments, an external monitor or other device can include a graphic display (e.g., displayed on a monitor) to display various parameters such as time period, frequency, intensity, etc. Thus, a microprocessor and memory can be provided in the external device in some embodiments.

The device of the present invention can be operated in a remote control mode wherein a Mobile App running on a smart phone provides via Bluetooth connectivity for energy application and biofeedback. Alternatively, the device can be operated in a computerized mode wherein software running on a personal computer provides for biofeedback.

The device can be operated with haptic and audio feedback. The connection to a phone, tablet or personal computer can be wireless via Bluetooth. That is, visual, audio and voice feedback, and prompts can be provided to facilitate device operation.

AI based learning is also contemplated for energy application to enhance therapeutic solutions/outcomes.

While the present invention has been described with reference to the specific embodiments thereof, it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

Where a range of values is provided, it is understood that each intervening value, between the upper and lower limit of that range is encompassed within the invention.

Although the apparatus and methods of the subject invention have been described with respect to preferred embodiments, which constitute non-limiting examples, those skilled in the art will readily appreciate that changes and modifications may be made thereto without departing from the spirit and scope of the present invention as defined by the appended claims.

It will be understood by those skilled in the art that the above particular embodiments are shown and described by way of illustration only. The principles and the features of the present disclosure may be employed in various and numerous embodiments thereof without departing from the scope and spirit of the disclosure as claimed. The above-described embodiments do not restrict the scope of the disclosure.

Additionally, persons skilled in the art will understand that the elements and features shown or described in connection with one embodiment may be combined with those of another embodiment without departing from the scope of the present invention.

Throughout the present disclosure, terms such as “approximately,” “about,” “generally,” “substantially,” and the like should be understood to allow for variations in any numerical range or concept with which they are associated. For example, it is intended that the use of terms such as “approximately,” “about” and “generally” should be understood to encompass variations on the order of 25%, or to allow for manufacturing tolerances and/or deviations in design.

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present disclosure.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

What is claimed is:

1. A therapeutic system comprising:
a) a generator for outputting energy at a first frequency; and
b) at least one probe electrically connected to the generator, the at least one probe having a user contacting surface;
c) wherein the generator transmits energy to a user at the first frequency via the at least one probe and energy from the user is returned to the generator via the at least one probe, and subsequently energy is transmitted back from the generator to the user via the at least one probe.

2. The therapeutic system of claim 1, wherein the at least one probe comprises first and second hand probes engageable by hands of the user.

3. The therapeutic system of claim 1, wherein the at least one probe comprises first and second foot probes engageable by feet of the user.

4. The therapeutic system of claim 1, wherein energy from the user sensed as unbalanced or disturbed is changed into a balanced state and balanced energy is sent back to the user via the at least one probe.

5. The therapeutic system of claim 4, wherein the generator includes a filter to change energy into the balanced state.

6. The therapeutic system of claim 1, wherein energy is transmitted continuously.

7. The therapeutic system of claim 4, wherein pulsed energy is transmitted.

8. The therapeutic system of claim 1, further comprising a data storage device for storing data relating to one or more of a) usage of the system; b) parameters during use of the system; and reactions by the user of the system.

9. The therapeutic system of claim 1, wherein the generator is activated by a remote device.

10. The therapeutic system of claim 1, further comprising a graphic display displaying parameters during use of the system.

11. The therapeutic system of claim 4, further comprising an outplate in communication with the generator, the outplate configured to communicate with a drinkable liquid in a container supported on the outplate, wherein the balanced energy is transmitted to the liquid in the container, the liquid providing a therapeutic liquid drinkable by the user.

12. The therapeutic system of claim 11, further comprising an inplate, the inplate configured to receive a substance or remedy, and the system detects a vibrational frequency of the substance or remedy placed on the inplate, wherein energy from the substance or remedy on the inplate is combined with the balanced energy and transmitted via the generator to the liquid in the container to create a therapeutic drink.

13. The therapeutic system of claim 12, wherein the combined energy is transferred to the user as the user is in contact with the at least one probe, thereby providing therapy to the user via the at least one probe and through the therapeutic drink.

14. The therapeutic system of claim 1, wherein the system uses scalar energy transmitting scalar waves.

15. The therapeutic system of claim 1, wherein balanced energy is transmitted to a drinkable liquid in electrical communication with the generator to create a therapeutic drinkable liquid.

16. A method of transmitting energy to a body of a user to improve health conditions, the method comprising:
a) outputting energy from a generator at a first frequency; and
b) transmitting the energy at the first frequency to the user via at least one probe electrically connected to the generator, the at least one probe having a skin contacting surface;
c) detecting via the at least one probe disturbed energy patterns in the user;
d) transmitting the disturbed energy to the generator;
e) filtering the disturbed energy in the generator to balance the energy; and
f) transmitting the balanced energy from the generator to the user via the at least one probe.

17. The method of claim 16, further comprising transmitting the balanced energy to a drinkable liquid to provide a therapeutic solution drinkable by the user.

18. The method of claim 17, further comprising detecting via a sensor a frequency of a substance or remedy and communicating the frequency of the substance or remedy to the generator communicating with a sensing device.

19. The method of claim 18, wherein the energy from the substance or remedy is combined with the balanced energy and transmitted to the user.

20. The method of claim 19, wherein the energy from the substance or remedy is combined with the balanced energy and transmitted to the drinkable liquid.
